(19) United States
(12) Patent Application Publication
(10) Pub. No.: US 2009/0310123 A1
(43) Pub. Date: Dec. 17, 2009

(54) Blood concentrate detector

(76) Inventor: Robert John Thomson, Warwick, RI (US)

Correspondence Address: Frost Controls, Inc., 51 Cromwell Avenue, Warwick, RI, 02889, US

(21) Appl. No.: 12/214,011

(22) Filed: Jun. 16, 2008

(51) Int. Cl. G01N 33/49

(52) U.S. Cl. 356/40

(57) ABSTRACT

A blood concentrate detector is disclosed for non-invasive, optical, detection of blood in translucent fluids, within translucent tubes, wherein the fluid is formed into a biconvex lens to 1^{st} disperse the light beam, being emitted through a 0.03 to 0.06 inch orifice, across the inner diameter of the tube, then 2^{nd} for that dispersed light to be refocused, via the fluid acting as a lens in the second half of the tube, and directed through a 0.030 to 0.06 inch aperture on the opposing side for detection purposes; whereas the detector measures the reduction in light energy do to the absorption by blood particles of light being emitted in the visible light range of 400 to 800 nm wavelength. Diffusing the light energy throughout the fluid creates an increase ratio of light energy absorption, whereby the electronic circuit can calculate the percentage of blood concentrate within the fluid.

### BACKGROUND

1. Field of Invention

The present invention relates to the non-invasive, optical detection of blood in translucent fluids. In particular, this invention relates to a system of interrelated optical devices, lenses and electronic circuit designed and deployed to detect the intrusion of blood in intravenous tubing for extracorporeal medical devices. More particularly, this invention relates to the detection of blood concentrates within the fluid More particularly yet, this invention relates to improved blood leak detector technology manifesting in increased reliability and accuracy These improvements include; liquid lens technology, proportional light energy absorption properties, electronic circuit stability as well as a means of self-monitoring proper installation of tubing and fluid flow.

2. Prior Art

Early model blood leak detectors used invasive optical means whereby they redirect the flow of dialysate (and blood) pass sensor(s) embedded in the intravenous tubing; ref U.S. Pat. Nos. 4,085,047; 4,017,190; 3,832,067. Because the solution and blood came in contact with the apparatus, it caused serious hygiene concerns, plus created ample work and costs in the cleansing process between patients. Shortly thereafter non-invasive optical detectors were employed that used the ‘angle of incidence’ theory for their mode of operation; whereby they tried to detect the change in angle of reflectance of dialysate vs. blood; ref U.S. Pat. No. 4,166,961. These devices could not detect small concentrates of blood particles within the dialysate solution. Still other blood leak detectors tried to detect the introduction of blood in dialysate solution by use of variance in reflectance properties of different wavelengths of light; ref U.S. Pat. No. 5,734,464. These were prone to drift, required frequent calibration and are susceptible to ambient light and electrical noise. Still other blood leak detectors attempted a similar procedure by using long and short wavelengths, then to electronically balance the two and “look” for a change in state of one of them; ref U.S. Pat. No. 4,181,610. These fell to the same short comings as above e.g. drift, frequent calibration, ambient light and electrical noise. Still others tried to overcome these shortcomings by damping their effects by comparing the signal outputs, from multiple samples, to know references stored in a software program; ref U.S. Pat. No. 5,670,050. Such software and hardware created significant cost to the device, plus was only masking the inherent shortcoming. Others employed single beam optics in the near infrared range, e.g. 800 to 930 nm wavelengths, that pass a narrow beam of light, e.g. 0.03 to 0.06 inch diameter, through intravenous tubing, whereby they need to squash the tubing in order for the light to penetrate the tube; ref. U.S. Pat. No. 7,230,687. Although an improvement over previous devices from the stand point of overcoming the shortcomings of drift, frequent calibrations and ambient conditions, these devices utilize a narrow field of view e.g. 0.03 to 0.06 inch beam width; thus fail to sample a significant portion of the dialysate to determine low concentrates of blood. Plus these devices employ light in the near infrared range that utilizes a contrast measurement—not a blood absorption measurement, of which this light range is susceptible to like contrast measurements from other contaminants; such as bubbles and micro bubbles. In addition, these devices require the use of a pop-up vane, which is an opaque material that blocks the light beam when the intravenous tubing is removed. This would indicate, to one skilled in the art, that the sensor can not detect the difference between the absence of the tube and a tube without fluid and/or a tube with clear fluid being present. The pop-up vane, being a mechanical device, is inherently subjected to failure; due to wear and clogging of its actuating mechanism, e.g. spring, rocker, etc. Plus, given the sticky nature of intravenous fluids and their tendency to leak, would require meticulous cleaning after each application. Still others have attempted utilizing laser light beam sources. These create an even tighter light beam path, which narrows the sampling of fluid being viewed, plus the laser light creates heat that can alter the characteristics the solution.

### Advantages

The present device overcomes the problems described above by creating a non-invasive detection means, whereby the sensor housing forms the translucent tubing and fluid into a biconvex lens to disperse a light beam across the majority of the fluid then to collect the dispersed light energy to a single point on the opposing side; thus being able to calculate the light absorption over a much wider viewing area than the projected beam path; typically 0.03 to 0.06 inch diameter. The sensor housing also shields the detector from the effects of ambient light, overcoming the problem of previous devices. The present invention also overcomes the problems associated with frequent calibrations, drift and electrical noise by way of achieving a strong signal to noise ratio. This is accomplished through the use of light that is the most susceptible to absorption by blood e g light in the visible spectrum—400 to 800 nm wavelength; in combination with dispersing this light energy over the majority of fluid within the tube, therefore allowing for the maximum level of energy absorption by the blood present. The use of light in the visible spectrum also overcomes the light scattering effects caused by bubbles within the fluid e.g. light in longer wavelengths is more susceptible to scattering by air bubbles. By achieving a high signal to noise ratio, the present device can reliable detect the states of:
absence of tubing (which negates the need for a pop-up vane and its associated problems)
presence of translucent tubing
presence of translucent tubing with the presence of translucent fluid
presence of small concentrates of blood within fluid
presence of higher concentrates of blood within fluid
presence of all blood within tubing.

By utilizing visible light produced from light emitting diodes (LEDs), creates little to no heat, thus alterations in the characteristics of the solution is avoided unlike blood leak detectors that employ laser or incandescent light.

### SUMMARY OF THE INVENTION

Recent developments in international medical equipment standards has required multiple detection levels of blood percentage within intravenous tubing for extracorporeal treatment systems; whereby the equipment is to react differently to differing concentrates of blood such as: indicate, annunciate, shut down. It is envisioned that the requirements will be further refined as the blood detection technology is further developed. In addition to having a blood concentrate detector determine the percentage of blood in solution over as wide a range as possible, it is also desirable to minimize adverse effects upon the sensor do to their environment; such as contact with solution/blood, leakage of solution/blood into mechanical parts, electrical noise interference, ambient light interference, etc. Plus the sensor should be designed in such a manner to avoid the need for calibration and should be easily cleaned without damage. The blood concentrate detector described here is believed to satisfy these requirements and to overcome the shortcomings of prior blood leak detectors.

In a first embodiment, the invention is a blood concentrate detector comprising a light source emitting light, in a wavelength range of 400 to 800 nm, to an opposing photo-detector with amplification circuitry, housed in an enclosure that can accept an intravenous tube.

A second embodiment of the invention is a blood concentrate detector comprising a housing having a slotted opening for insertion of the intravenous tube, wherein the slot opening is smaller than the nesting position of the tube, where as the housing acts to secure the tube in place and at the desired optical path between the light emitter and photo-detector receiver.

A third embodiment of the invention is a blood concentrate detector comprising a housing having a nesting position that forms, and/or allows the tube to form, a biconvex lens when filled with translucent fluid.

A forth embodiment of the invention is a blood concentrate detector comprising a housing having embedded optical filters for the light to traverse through the inner wall of the nesting area and through the opposing inner wall of the nesting area to the receiver.

A fifth embodiment of the invention is a blood concentrate detector comprising an analog circuit that can accurately measure the light intensity of: absence of tubing, presence of translucent tubing, presence of translucent tubing with the presence of translucent fluid, presence of small concentrates of blood within fluid, presence of higher concentrates of blood within fluid, and the presence of all blood within tubing.

### BRIEF DESCRIPTION OF DRAWINGS

FIG. 1 is a front view of the blood concentrate detector housing.

FIG. 2 is a front view of the blood concentrate detector housing with intravenous tubing in place, depicting the optical path.

FIG. 3 is a front view of the blood concentrate detector housing with intravenous tubing with fluid in place, depicting the change in optical path.

FIG. 4 is a front view of the blood concentrate detector housing, depicting embedded optical filters.

FIG. 5 is graph of signal transmission levels for states of interest.

### DRAWINGS—LISTING OF REFERENCE NUMERALS

100—Light source emitter
102—Photo-detector receiver
104—Printed Circuit Board (PCB)
106—Intravenous tubing cavity (nesting area)
108—tube slot entry and capture
110—Housing
112—Connection for power and output
114—Light source aperture
116—Photo-detector orifice
200—Conical expansion of light beam
202—Focus of part of the beam
204—Intravenous tube without fluid present
300—Intravenous tube with fluid present
302—Conical expansion of light beam with lens effect
304—Focus of light energy with lens effect
306—Biconvex lens effect
400—Embedded optical filters
500—Signal level without tube present
502—Signal level with tube present
504—Signal level with tube and fluid present
506—Signal level with tube, fluid and small concentrate of blood present
508—Signal level with tube, fluid and higher concentrates of blood present
510—Signal level with tube and all blood present

### DETAILED DESCRIPTION OF THE INVENTION

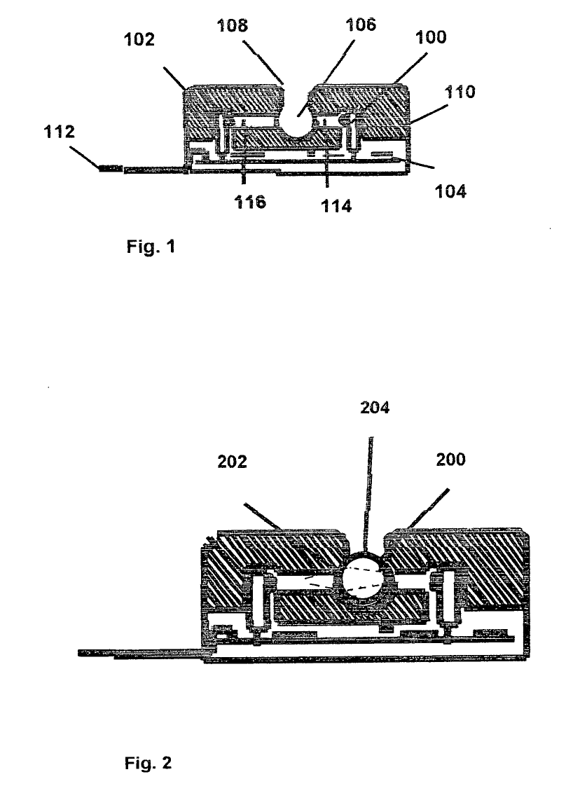

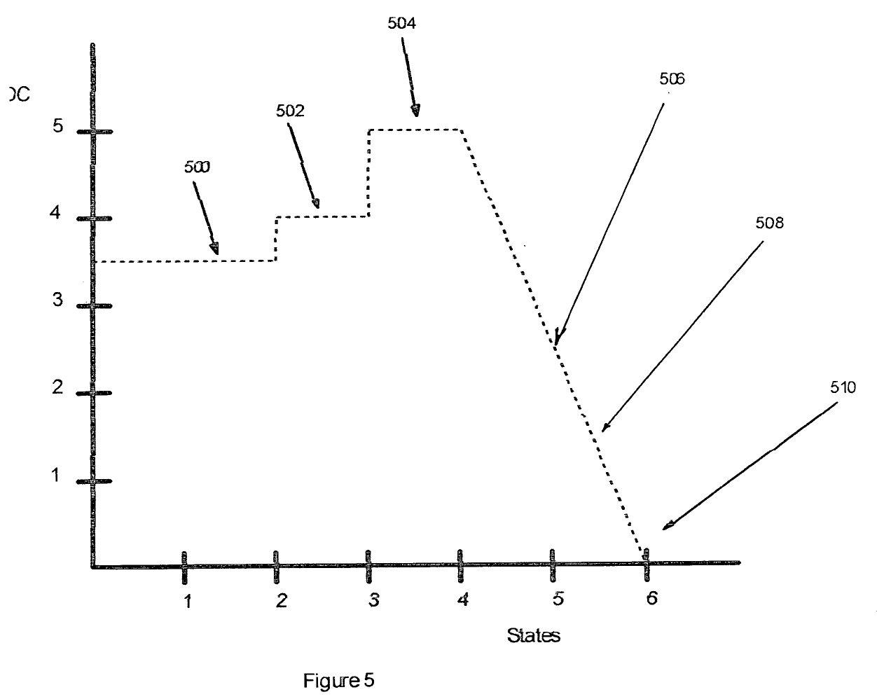

FIG. 1 shows a perspective view of the blood concentrate detector used in connection with intravenous tubing for extracorporeal treatment system whereas it is desirable to detect the malfunctioning of equipment and/or the misapplication of apparatus used in the medical treatment system. Such equipment malfunctions include, but are not limited to, pump stoppage, pump reverse, filter leakage, filter breakage, circulation interference, etc. Such misapplication of apparatus include, but are not limited to: intravenous tubing not properly inserted or pulled out of the tube cavity (e.g. nesting area, optical pathway); air in tubing due to fluid leakage, dispenser empty, etc.; intravenous tubing kinked; et. FIG. 5 depicts the various states that will manifest as a result of the various malfunctions and/or misapplications described. FIG. 1 depicts an optical system to detect these various states, by way of generating a light 100 and transmitting said light through an aperture 114, across an open space 106, through an orifice 116 to a photo-detector 102, where as said photo-detector transmits an electrical signal to the circuitry 104 for signal manipulation and output 112 to the controlled or controlling equipment. Said output connection 112 also can, but does not necessarily have to, serve as a port of entry for input power. The open space 106 is the intravenous tube cavity and is designed in such a way to ensure the tube maintains a circular pattern and is nested in front of the light beam pathway. The flexible intravenous tubing is compressed through the tube slot entry 108 and is captured by the tube cavity 106 to maintain its orientation to the light pathway. The preferred optical path, but not required optical path, is for the centerline of the light beam 114 to be injected in or near the diameter of the tubing 204 whereby the greatest biconvex lens effects of the fluid 306 will take place. To direct the light beam, a tunnel effect is created by channeling the light through an aperture 114 of 0.030 to 0.060 inch in diameter. The preferred aperture diameter is a function of the tube diameter. Said light beam 100 is generated by a light emitting diode (LED) in the visible light spectrum i.e. range of 400 to 800 wavelength. The visible light spectrum is found to be more greatly absorbed, by organic matter, than light in other ranges such as; near infrared, far infrared and ultraviolet. Blood, being an organic matter, absorbs light energy in the visible light spectrum in greater proportionality than light energy in other wavelengths. Utilizing light in the 400 to 800 nm wavelength provides for better contrast in signal strength between fluid and fluid with small concentrates of blood. In addition, utilizing light in the 400 to 800 nm wavelength provides for near linear decrease in signal strength proportionate to the percentage of blood in the fluid. LED light is preferred over other light sources, such as laser or incandescent, because it does not produce heat. Heat can have negative effects on substances used for patient care.

As the light beam passes through the aperture 114 it traverses in a conical pattern across the tube cavity 106 with a percentage of the light beam entering the photo-detector orifice 116 and traveling to the photo-detector 102. The conical expansion of the light beam over the entire space of the tube cavity 108 is a function of the tube being absent FIG. 1 and depicts state 1 of FIG. 5 with the signal level without tube present 500. The photo-detector signal passes to the circuitry for signal manipulation and output.

FIG. 2 shows said blood concentrate detector with intravenous tubing present 204 and without fluid present. The conical expansion of the light beam 200 acts in a similar manner as described above, but the inner wall of the opposing side of the tube acts as a lens and gathers a larger percent of the light beam diameter and redirects that light 202 through the photo-detector orifice 116 towards the photo-detector receiver 102. FIG. 2 depicts state 2 of FIG. 5 with the signal level with tube present 502. The photo-detector signal passes to the circuitry for signal manipulation and output.

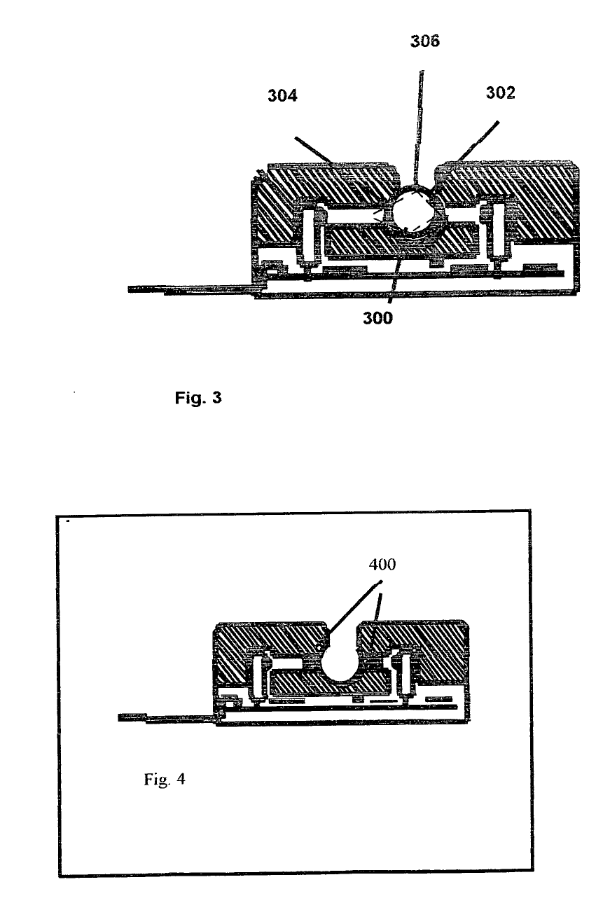

FIG. 3 shows said blood concentrate detector with intravenous tubing present and with fluid present 300. The intravenous tubing cavity forms, the otherwise imperfectly shaped tube, into a biconvex lens 306 when filled with translucent fluid. The biconvex lens effect 306 of the fluid, in the first half of the tube, expands the conical angle of the light beam 200 and disperses the light energy to a wider angle, covering the inner diameter of the tube. The inner walls of the tubing act as a reflector to trap and scatter the light energy throughout the fluid, providing nearly 100% coverage of the fluid in the inner diameter of the tube at the plane of detection. By dispersing the light energy over the fluid within the entire inner diameter creates the maximum amount of light energy absorption by blood particles when blood particles exist 506. The fluid in the second half of the tube acts as the second half of the biconvex lens 306 and focuses the light energy 304 dispersed throughout the fluid to the photo-detector orifice 116 towards the photo-detector receiver 102. FIG. 3 depicts state 3 of FIG. 5 with the signal level with tube and fluid present 504. The photo-detector signal passes to the circuitry for signal manipulation and output.

FIG. 4 shows said blood concentrate detector with optical filters 400 embedded in said intravenous tubing cavity 106 at said light source aperture 114 and at said photo-detector orifice 116. The tunnel from the photo-detector orifice 116 to the photo-detector prevents ambient light from directly striking the photo-detector. Said optical filters 400 are used to filter out extraneous light in wavelengths other than the chosen light range to further shield the photodetector from the effects of ambient light. Said tube slot entry and capture 108 also acts as a shroud to block the effects of ambient light. It is envisioned that further protection from ambient light, if needed, could be achieved through the use of a modulated light source and de-modulated receiving circuit. This is a well known technique to those skilled in the art. Said embedded optical filters 400 also functions to prevent the ingress of foreign matter into the optical pathway and circuitry.

FIG. 5 depicts the various states of said blood concentrate detector with; state 1 representing the signal level without tube present 500, state 2 representing the signal level with tube present 502, state 3 representing the signal level with tube and fluid present 504, state 4 representing the signal level with tube, fluid and small concentrate of blood present 506, state 5 representing the signal level with tube, fluid and higher concentrates of blood present 508, and state 6 representing the signal level with tube and all blood present 510. Said state 4 represents a blood concentrate of approximately 3% of the fluid content while said state 5 represents a blood concentrate level of approximately 25%. The current state of the art requires detection systems to react to 25% blood concentrate levels. The analog signal of said blood concentrate detector at the 3% blood concentrate level 506 and 25% blood concentrate level 508 is far below that of the various states of illumination, i.e. states 1 through 3. This provides for accurate and reliable detection of blood in fluids for extracorporeal treatment systems. The significant incremental steps of the analog signal for the various states of illumination i.e. states 1, 2 and 3, provides for accurate and reliable detection of tube not present 500, tube present 502, and tube and fluid present 504.

While the above descriptions contain much specificity, these should not be construed as limitations on the scope, but rather as an exemplification of several preferred embodiments thereof Many other variations are possible. For example, it is envisioned that; multiple light emitters and photo-detectors could be employed for redundancy, blood flow rate measuring, etc. or signal manipulation could be done extraneous to the sensor(s) or modification to the tube nesting area could be achieved or analog signal measurements could be converted to digital signals or embedded optical filters could be attached to the optical elements in lieu of the housing or fewer or more increments in signal levels could be disclosed, etc.

Accordingly, the scope should be determined not by the embodiment(s) illustrated, but by the appended claims and their legal equivalents.

We claim:

1. A blood concentrate detector comprising:
a. a light source to create visible light in the wavelength of 400 to 800 nm;
b. a means to project said visible light through an aperture, across a tubular cavity, through an orifice to a photo-detector with said tubular cavity having a circular shape and slot opening to receive and hold in place a tube for liquid and;
c. a means to detect states in said visible light as the light passes through; air, translucent tube, translucent tube with translucent fluid, translucent tube with translucent fluid with various concentrates of blood,
wherein said detector indicates the degree of malfunction of equipment or misapplication of apparatus used in extracorporeal medical treatment systems.

2. The blood concentrate detector in claim 1 wherein the means to create said light is by light emitting diode in the green light spectrum range.

3. The blood concentrate detector in claim 1 wherein the means to detect variations in said light is by phototransistor.

4. The blood concentrate detector in claim 1 wherein said tubular cavity shrouds the photo-detector from ambient light.

5. The blood concentrate detector in claim 1 wherein the light source aperture and photo-detector orifice are sealed with optical filters flat prevent the ingress of foreign matter and filter out light in extraneous wavelengths

6. A blood concentrate detector comprising: a housing having an opening to receive a tube for liquid, a tubular cavity for said tube to nest, a visible light source on one side of tubular cavity, a photo-detector on opposing side of said tubular cavity; wherein said tubular cavity forms said tube for liquid into a liquid biconvex lens when translucent fluids are present.

7. The blood concentrate detector in claim 5 wherein said tubular cavity forms said tube for liquid into a convex lens when translucent fluids are not present.

8. The blood concentrate detector in claim 5 wherein the light beam path is at or near the diameter of said tube for liquid whereby the said liquid biconvex lens first disperses the light energy of said light beam path throughout the said translucent fluid then secondly collects the dispersed light energy and focuses said dispersed light energy towards the photo-detector.

9. A method for detecting the states of:
a. the absence of a translucent liquid carrying tube;
b. the presence of a translucent liquid carrying tube;
c. the presence of a translucent liquid carrying tube with translucent fluid and;
d. the presence of a translucent liquid carrying tube with translucent fluid with varying percents of blood within said translucent fluid;
using a blood concentrate detector having a visible light source, a photo-detector, a tubular cavity, an opening to insert said translucent liquid carrying tube, and circuitry; wherein said method measures the variations in light energy of said visible light source created by the optical effects of said translucent liquid carrying tube when, absent, present, or present with translucent fluids; and created by the light energy absorption properties of blood to light in the visible spectrum.

10. The method in claim 8 comprising: a light emitting diode for said visible light source in the 490 to 570 nm wavelength for proportional light energy absorption by blood based on the percentage of blood present in fluid.
